(19) United States
(12) Patent Application Publication
(10) Pub. No.: US 2021/0225496 A1
(43) Pub. Date: Jul. 22, 2021

(54) SYSTEMS AND METHODS FOR SCHEDULING DELIVERY OF HEALTHCARE SERVICES

(71) Applicant: AccentCare, Inc., Dallas, TX (US)

(72) Inventors: David Vontress, Dallas, TX (US); Dan Kinn, Dallas, TX (US); Biju Phillips, Dallas, TX (US); Jonathan Bard, Dallas, TX (US)

(21) Appl. No.: 17/151,428

(22) Filed: Jan. 18, 2021

Related U.S. Application Data
(63) Continuation-in-part of application No. 17/139,648, filed on Dec. 31, 2020.
(60) Provisional application No. 62/962,783, filed on Jan. 17, 2020.
(60) Provisional application No. 62/962,783, filed on Jan. 17, 2020.

(51) Int. Cl. G16H 40/20; G06Q 10/06; G06Q 10/04; G06F 16/906

(52) U.S. Cl. CPC G16H 40/20; G06Q 10/063118; G06F 16/906; G06Q 10/063112; G06Q 10/047

(57) ABSTRACT

Systems and methods are described herein for scheduling the on-site delivery of health care services across a wide geographic area.

### RELATED APPLICATIONS

This application is a continuation in part application of U.S. application Ser. No. 17/139,648, filed Dec. 31, 2020, which claims the benefit of U.S. Patent Application No. 62/962,783, filed Jan. 17, 2020.

This application claims the benefit of U.S. Patent Application No. 62/962,783, filed Jan. 17, 2020.

### TECHNICAL FIELD

The disclosure herein involves scheduling deployment of health care providers for delivery of health care services.

### BACKGROUND

In a majority of settings, rehabilitative, nursing and occupational therapy services are provided by skilled clinicians at either healthcare facilities or the patients' home. Depending on the organization providing the care, the clinicians may be company employees, independent contractors, or outside agency workers. Facilities include hospitals, nursing homes, clinics and assisted living centers, and may be located throughout a wide geographic area. There is a need to develop systems and methods for scheduling deployment of health care providers for delivery of health care services across such wide geographic area.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE DRAWINGS

FIG. 1 provides a patient clustering demonstration, under an embodiment.

FIG. 2 provides a patient clustering demonstration, under an embodiment.

FIG. 3 shows a route comparison for visit-to-day assignment, under an embodiment.

FIG. 4A shows an input set of points, under an embodiment.

FIG. 4B shows a convex hull created from a set of points, under an embodiment.

FIG. 5A shows a set of points on a two dimensional plane, under an embodiment.

FIG. 5B shows a convex hull, under an embodiment.

FIG. 5C shows a plurality of convex hulls, under an embodiment.

FIG. 5D shows a plurality of convex hulls, under an embodiment.

FIG. 5E shows a plurality of convex hulls, under an embodiment.

FIG. 5F shows a plurality of convex hulls, under an embodiment.

FIG. 5G shows a plurality of convex hulls, under an embodiment.

FIG. 6 shows inner and outer hulls, under an embodiment.

FIG. 7 shows a connection of inner and outer hulls, under an embodiment.

FIG. 8 shows possible splits and pairings of the inner and outer hulls shown in FIG. 6, under an embodiment.

FIG. 9 shows output of a scheduling application, under an embodiment.

FIG. 10 illustrates a cost matrix, under an embodiment.

FIG. 11A shows a method for scheduling delivery of health care services, under an embodiment.

FIG. 11B shows a method for scheduling delivery of health care services, under an embodiment.

### DETAILED DESCRIPTION

### 1. Introduction

### 1.1 Problem Description

The purpose of the systems and methods described herein help home health companies construct weekly schedules for their workforce. This activity generally takes place several days prior to the beginning of each work week and is governed by a combination of known and unknown demand for services, and a wide range of logistical, legal, policy, and travel constraints.

Smart Scheduler is a sequence of optimization models for the purpose of assigning a given set of patient visits to clinicians at minimum marginal cost while fulfilling all patient care constraints.

The marginal cost of a visit is calculated as the sum of the marginal cost of each clinician-to-visit assignment, and, generally, can consist of many cost factors (i.e. travel time, continuity of care, and labor cost). Patient care constraints ensure that quality care is given to patients, and, generally, restrict the set of clinician-to-visit assignments that can be made based on capacity and care requirements.

The algorithm is composed of several stages to reduce computational complexity and solve time as well as to allow parallelization of some components of the algorithm. The stages, in brief, are:

1. Pre-processing of input data: This includes steps to generate the marginal cost matrix, the clustering of nearby patient visits, and load balancing across the scheduling horizon.
2. Clinician-to-visit assignment: Assign each visit to a clinician in the least-cost manner, ensuring that capacity and eligibility constraints are met. These constraints, among other things, include ensuring that clinicians are not overloaded on any individual day or week, ensure that clinicians are assigned visits within their skill level, and that patients are assigned compatible clinicians.
3. Clinician daily routing: For each clinician, determine 1) which day of the week to complete each visit, and 2) the order to conduct each day's visits.

### 2. Preprocessing

Preprocessing primarily entails reading input data from the source database(s) and formatting the data for the optimization models. The model requires input data for the visits on the visit horizon as well as the clinicians that can be scheduled. Additional parameter array and matrix inputs are required as described in the models. Other preprocessing steps are described below.

### 2.1 Marginal Cost Generation

As described above, Smart Scheduler seeks to minimize the total marginal cost of visiting each patient in the assignment horizon while ensuring patient care constraints are met. We seek only to minimize marginal costs in short-term planning because, in the short-term, the fixed costs cannot be adjusted by our scheduling decisions.

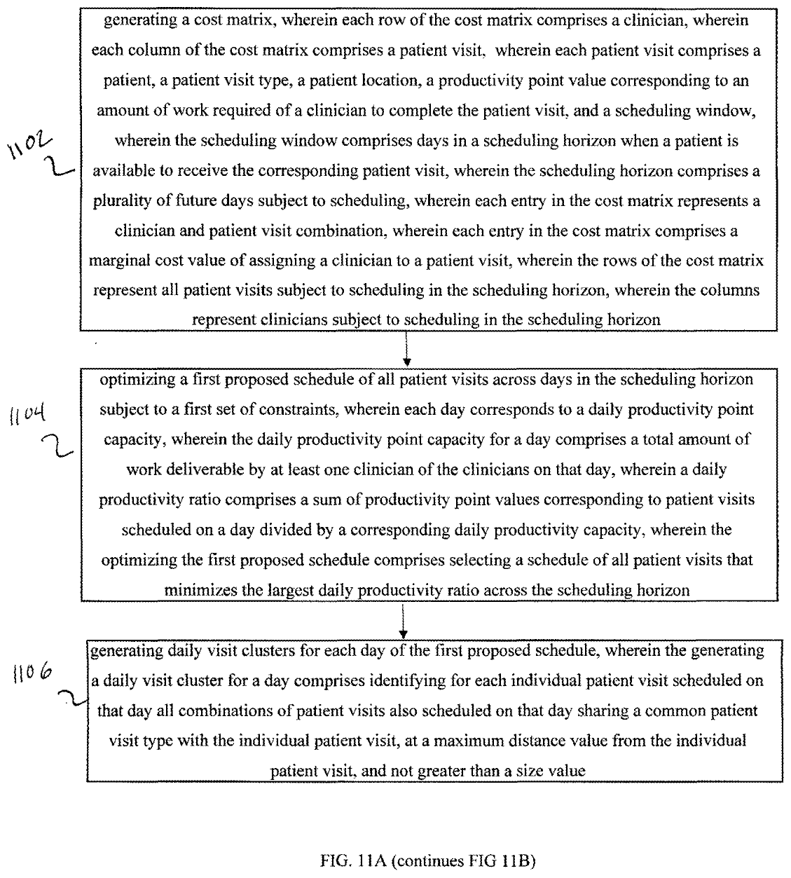

A large part of the pre-processing stage is to generate the marginal cost matrix that is the core component of our objective function. The marginal cost matrix, C, is a |W|×|I| matrix, where W is the set of clinicians and I is the set of patient visits. Each entry in the cost matrix, c_{w,i} represents the marginal cost of assigning clinician w to patient visit i. FIG. 10 illustrates the cost matrix, under an embodiment.

The marginal cost matrix is a composite matrix of the different cost components that go into a visit. In our case, the cost components are the marginal cost of labor, the cost traveling from the clinician's residence and the location of the patient visit, and a continuity of care penalty cost that motivates the algorithm to keep continuity between patients and their clinicians.

### Marginal Labor Cost

Marginal labor cost is the additional cost incurred by assigning visit i to clinician wₛ. This cost is dependent on the payment method of the clinician. Salaried clinicians, for instance, have a marginal cost of $0 for each visit they complete, since they are paid a fixed salary regardless of the number of visits they complete (excluding overtime, which is handled separately). On the other extreme are contractors, which are paid a fixed-rate for each visit they complete. In this case, the marginal cost of assigning contractor we to visit i is the contractor's rate for that visit type.

### Travel Cost

Travel cost is an important piece of the marginal cost of assigning visit i to clinician w, for several reasons. Clinicians generally get compensated in some form for the miles they travel or time spent on the road. Further, the more time clinicians spend driving, the less time they have to conduct patient visits. Finally, high average travel times between patients can contribute to clinician burn-out and is a main reason for turnover in the industry.

Because the Smart Scheduler algorithm is broken down into two stages, we cannot optimize based on actual route travel times. Routes are generated in Stage 2, after the assignment takes place in Stage 1. Instead, we use travel time between the clinician's residence and the patient visit location as a proxy for actual experienced travel times. Travel time data between points is imported from a third-party vendor. This travel time must then be converted into a dollar value using some conversion formula, i.e. $1 per minute of travel.

### Continuity of Care Cost

Continuity of care is a metric that measures how frequently each patient sees the same clinicians on recurring visits. The metric is a key component of patient and clinician satisfaction and is a contributor to successful patient outcomes. Under one embodiment, a continuity of care penalty is zero if a clinician is assigned to a patient visit when that clinician has conducted visits for that patient in the past. If a new clinician is assigned to such patient, the continuity of care penalty is $40.00. While this is not a direct cost that is borne by the business, we must assign a cost value to continuity of care in order for the metric to enter the objective function and thus motivate the algorithm to assign matches with high continuity of care.

### 2.2 Nearby Visit Clustering

Frequently, patients will be located in the same or similar locations. This is the certainly case with senior living facilities and senior communities, but instances of patients with similar care needs living in nearby locations occur frequently in dense geographic networks even outside of these situations. It is beneficial to assign nearby patients to the same clinician when clinically feasible, since it reduces travel time.

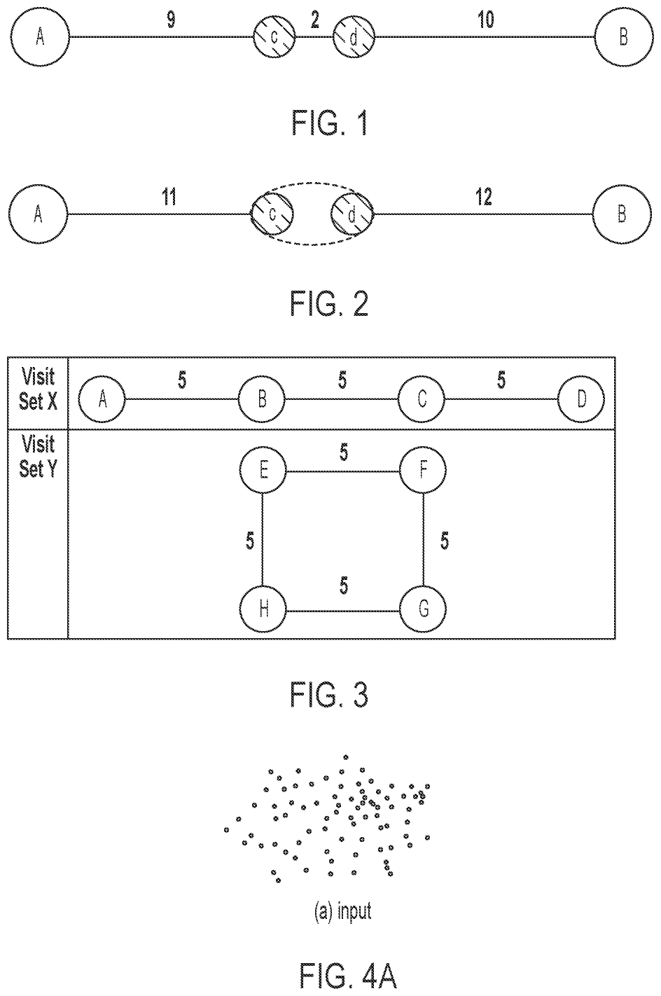

However, the generalized assignment model does not capture this benefit, since the model measures distance from the clinician's residence to the patient location, not the distance between patient locations. To rectify this, we create visit clusters of nearby visits that occur on the same day, to incentivize the algorithm to assign these visits to the same clinician. This is best demonstrated through an example. Consider the simple network in FIG. 1, in which there are two clinicians (A and B) and two patient visits (c and d). In this simplified example, we assume that clinicians have unlimited capacity and we ignore labor and continuity of care costs.

Without visit clustering, the assignment model assigns visit c to clinician A and visit d to clinician B at a total cost of 19 units. The assignment model does not assign visit d to clinician A, because doing so would cost a total of 20 units (9+11).

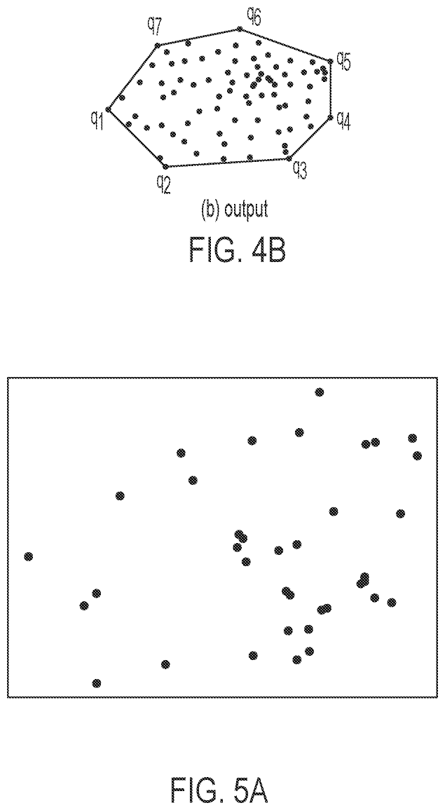

However, if we allow visits c and d to be clustered together, we can significantly reduce total travel time. FIG. 2 shows the costs for each clinician of visiting the cluster of patients. For clinician A, this cost is 11, since A would take the route A→c→d to visit both patients, at a cost of 9+2=11.

### Technical Implementation

Technically, the clusters are created as follows:

Let J be the set of all visit clusters. Let A_{d} be the set of visits that occur on day d. Let τ be the furthest intra-cluster distance. Let K be the maximum cluster size.

The first |I| clusters of J are all one-visit clusters containing only visit i.

For each day d in D:

For each visit i in A_{d}:
Let Ā_{d,i} be the set of visits that occur on day d and that are the same service code as visit I and are within i of visit i's location. Add to J each combination of visits
in Ā_{d,i} that is of maximal size K and not already in J. Each combination should also include visit i.
   The cost of assigning each visit cluster j to clinician w is then the sum of cost of assigning each visit i∈j to clinician w, less the duplicated travel time saved by clustering the visits.

### 2.3 Load Management

Patient visits occur throughout the week, as dictated by each patient's plan of care. Generally, the visits will not be perfectly distributed across the scheduling horizon (i.e. there may be 50 visits on Monday, 35 on Tuesday, 45 on Wednesday, etc.). However, from a labor scheduling perspective, the ideal case is an even distribution of visits across the week. This reduces the need for expensive variable cost labor (contractors and PRNs) on overloaded days, and reduces the underutilization of fixed cost labor (employees) on under-loaded days. Fortunately, there is some flexibility in the schedule date of some visits (between 0 and 2 days), which allows us to shift some visits around to improve weekly balance. We do this using a custom assignment model.

### 2.3.1 Model Inputs

### Sets and Indices

I Set of all patient visits on the scheduling horizon.
D Set of days on the scheduling horizon.
Λ Set containing lists of visits for the same patient. For example, if visits i₁ and i₂ both belong to patient p, then λₚ=(i₁,i₂).

### Parameters and Constants

pᵢ Productivity points for visit i.
δ_{d} Productivity point capacity on day d
γᵢ: Set of days that visit i can be scheduled on.

### Decision Variables

x_{i,d} Binary decision variable, 1 if visit i is assigned to day d
z₀ Non-negative decision variable, which represents the demand:supply ratio for the day with the highest demand:supply ratio

### 2.3.2 Load Management Optimization Model

The model seeks to minimize the highest demand:supply ratio. This forces the model to move visits from relatively overloaded days to relatively under-loaded days. This model assumes a short planning horizon, since it only minimizes the largest daily ratio.

Minimize z₀ (0a)

Minimize the decision variable z₀, which is the largest daily demand:supply ratio.

### Subject to:

The decision variable z₀ is greater than the largest daily demand:supply ratio.

$${z}_{0} \geq \frac{1}{{δ}_{d}} * {{\sum }_{i ∈ I}}_{ } {p}_{i} {x}_{i , d} ∀ d ∈ D \qquad ( 0 b )$$

Each visit is assigned to exactly one day

Σ_{d∈D}x_{i,d}=1∀i∈I (0c)

Visits cannot be assigned to days outside of their flexibility window

Σ_{d∉γ}_{ᵢ}x_{i,d}=0∀i∈I (0d)

Two visits for the same patient cannot be assigned to the same day

Σ_{i∈λ}x_{i,d}≤1∀λ∈Λ,d∈D (0e)

### 3. Clinician-to-Visit Assignment

Clinician-to-visit assignment is the first stage optimization model that assigns each visit on the scheduling horizon to a clinician in the least-cost manner. In this stage, patient visits for the planning horizon are assigned to clinicians. As stated in the problem description, the goal of the model is to minimize the total marginal cost, which is the sum of travel cost, continuity of care cost, and marginal labor cost. Given these costs, our objective is to maximize total score by assigning all visits to clinicians while ensuring that no clinician is overloaded with daily visits.

### 3.1 Model Inputs

### Sets and Indices

W Set of all clinicians
I Set of all patient visits for the upcoming week.
J Set of all visit clusters
D Set of all days for the upcoming week.

### Parameters and Constants

c_{w,j} Cost of assigning visit cluster j to clinician w.
pᵢ Productivity points for visit i.
η_{w,d} Productivity point capacity for clinician w on day d.
A_{d}: List of all visits occurring on day d.
Ψᵢ: Set of visits clusters that include visit i
φ_{w}: Set of all visits that can be completed by clinician w.

### Decision Variables

x_{w,i} Binary decision variable, 1 if clinician w is assigned to patient visit i
z_{w,j} Binary decision variable, 1 if clinician w is assigned to patient visit cluster j

### 3.2 Optimization Model

The model seeks to minimize the total cost across all clinician-patient-visit assignments. The first constraints (1b) ensures that each clinician is not assigned more productivity points than they can handle each day. If worker w is on vacation on day d, then w's capacity, η_{w,d}, is set to 0. The second constraint (1c) ensures that all visits are assigned to exactly one clinician, such that there are no unassigned visits.

Constraint 1d links visit assignments with visit cluster assignments. It requires that, if visit cluster j is assigned to worker w, then all of the visits that are part of cluster j are also assigned to worker w. In this way, this linkage constraint ties together the decision variables z from the objective function with the decision variables x that appear in the constraints.

Constraint 1e accounts for the differences in skill levels required for visits. There are numerous skill constraints in home healthcare to meet certain legal, ethical, and quality of care constraints; this constraint ensures that all these criteria are met for each visit.

Constraints 1f and 1g simply state that the decision variables are binary: no partial assignments can be made for any visit.

Minimize Σ_{j∈J}Σ_{w∈W}c_{w,j}z_{w,j} (1a)

Minimize the total cost of clinician-to-patient-cluster assignments

### Subject to:

Each clinician is assigned no more than their daily productivity point capacity

Σ_{i∈A}_{_{d}}pᵢx_{i,w}≤η_{w,d},∀w∈W (1b)

Each visit is assigned to exactly one clinician

$${\sum }_{w ∈ W} {x}_{i , w} = 1 , ∀ i ∈ I \qquad ( 1 c )$$

If visit i is assigned to clinician w, then there must also be a visit cluster containing visit i assigned to clinician w.

Σ_{ψ∈Ψ}_{ᵢ}z_{w,ψ}≥x_{w,i},∀w∈W,i∈I (1d)

Clinicians are not allowed to complete visits which they do not have the skills to complete.

Σ_{∉φ}_{_{w}}x_{i,w}=0,∀∈W (1e)

The binary decision variable x determines whether visit i is assigned to clinician w.

x_{i,w} binary ∀i∈I,w∈W (1f)

The binary decision variable z determines whether visit cluster j is assigned to clinician w.

z_{w,j} binary ∀w∈W,j∈J (1g)

### 4. Daily Clinician Routing

Once visits are assigned to clinicians, we must 1) determine which day each visit will take place on, and 2) the daily routes each clinician will take to visit each assigned visit. The daily routing is broken into these two steps to reduce complexity and thus improve solve times. Since daily clinician routing must be run for each clinician, and the goal is to generate a solution in under 30 minutes of run time, merging these steps into a single optimization model was too complex.

The two steps of the daily clinician routing phase are described separately, below. Note also that at this phase of Smart Scheduling, each clinician's route is independent of all other clinician's routes, and hence this stage can be run in parallel at the individual clinician level.

### 4.1 Visit-to-Day Assignment

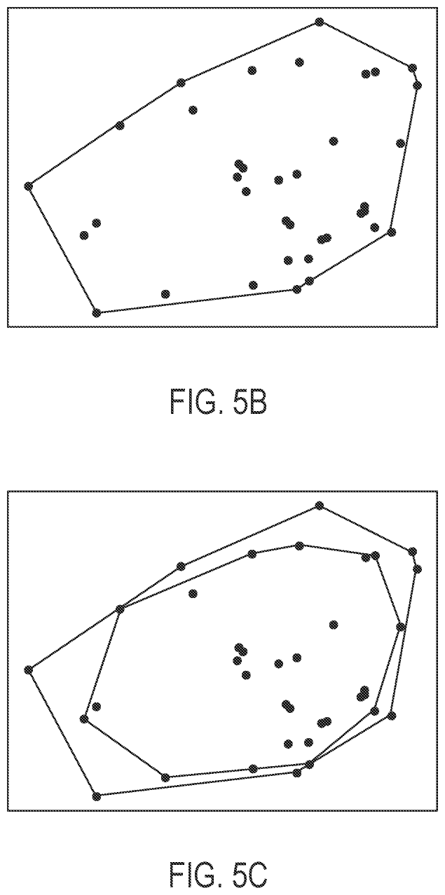

The goal with the visit-to-day model is to minimize the distance between the visits assigned to each day of the week. We model this by minimizing the sum of the distance between each visit and the centroid (geometric median) of each day's visits. Using this as our objective has two benefits: 1) generally, visits that are closer together will have short routes between all visits, and 2) visits that are clustered close together will incur smaller time penalties when/if clinicians have to reschedule patients throughout the day. To see why the second benefit is important, consider the example in FIG. 3 showing a route comparison for visit-to-day assignment

Assume that we can assign Clinician A either set of patient visits for a given day. We see that either assignment has a shortest route distance of 15 units (either A→B→C→D or E→F→G→H). However, let's assume that the second patient on the route (B or F) has a morning doctor's appointment and has to delay her visit by an hour. Hence, a clinician is forced to rearrange her schedule, such that A→C→B→D or E→G→F→H becomes the necessary route after rearranging the schedule. The route distance for Visit Set X then becomes 25, and for Visit Set Y becomes 19.14. This reschedule is much less costly in Visit Set Y, because Visit Set Y has a closer intra-patient distance. Reschedules are frequently required in practice, making this an important facet of daily assignment.

There are numerous types of visits, each with its own characteristics and clinical requirements. From a scheduling perspective, one of the most important characteristics is the amount of scheduling flexibility the visit type allows. This flexibility allows the movement of visits between 0-2 days around the original target visit days, depending on the visit type. This allows us to shift the visit dates on some visits when doing so reduces a clinician's total travel time. Visit-to-day assignment must also obey each clinician maximum daily capacity constraint, which prevents overloading a clinician on any day.

### Model

Minimize the sum of the intra-cluster distances across all day clusters.

Minimize Σ_{d∈D}Σ_{i∈I}c_{d,i}x_{d,i} (2a)

### Subject to:

Each day is assigned no more than an upper threshold of productivity points.

Σ_{i∈I}x_{d,i}≤η_{d}∀d∈D (2b)

All visits are assigned to exactly one day.

Σ_{d∈D}x_{d,i}=1∀i∈I (2c)

The binary decision variable determines whether visit i is assigned to day d.

x_{d,i} binary ∀d∈D,∀i∈I (2d)

### The Iterative Process

The above model is run in an iterative fashion until no further improvements are made to the objective function (or, until the number of iterations reaches an arbitrary upper threshold). The model is then run to obtain the first assignments of patient visits to days. Given these assignments, each day's centroid is updated to the geometric median of the visits assigned to that day. Because the geometric median minimizes the intra-cluster distance for each cluster, this necessarily is an improvement of our objective (unless the visits are already located at the geometric medians of their clusters, in which case the improvement is 0 and the algorithm terminates).

Given these updated daily centroids, the assignment model is then run again to create new assignments. Again, the centroid locations are updated to the geometric medians of their new clusters. This process is repeated until no improvement to the objective is made between steps.

1. Initialize |D|, the number of days
2. Initialize a set Ω, indexed by d∈D. Each ω_{d} ∈Ω represents the geographic center of the visits that are assigned to day d. Since initially no visits are assigned to any days, each ω_{d} ∈Ω is randomly assigned a point within the minimum bounding box of all visits.
3. Using Ω as the cluster centroids, assign patient visits to days of the week using the Visit-to-Day assignment model described in Section 4.1.
4. With the output from Step 3, update each ω in Ω to be the geographic center of the visits assigned to ω, using the geometric median.

a. If Ω is unchanged from the prior step (or alternate stopping criteria are met), the assignment is complete.

b. Else, return to Step 3.

### 4.2 Daily Routing

Once a clinician's visits are assigned to days of the week, the remaining item is to develop optimal routes for each day. The goal is to minimize the travel time incurred when visiting each patient. This is akin to The Traveling Salesman (TSP) problem, a well-studied problem in operations research.

Since a route must be developed for each day for each clinician, the number of daily routing problems is quite large, |D|*|W|. As such, a quick-solving heuristic was developed for solving small routing problems quickly. The solution is described below. It should be noted that any fast-solving TSP heuristic can be substituted, and the following heuristic is not exclusive to the home healthcare industry.

An optimal route must be designed that starts from the clinician's residence, visits each patient, and returns to the clinician's residence at the end of each day. The route should minimize total distance traveled, and is a traditional symmetric traveling salesman problem, which has been extensively studied and is NP-hard. The following heuristic was developed to solve the problem in polynomial time.

### Traveling Salesman Problem Heuristic

The following heuristic is used to solve a traditional symmetric traveling salesperson problem. The problem statement is as follows: a traveling salesperson (or, in our case, a clinician), leaves home and needs to visit each of n clients (visits) exactly once prior to returning home. The salesperson (clinician) seeks to minimize the total distance travelled across all visits. For simplicity of computation, we assume that distances are symmetric; that is, the distance from Patient A to Patient B is the same as the distance from Patient B to Patient A.

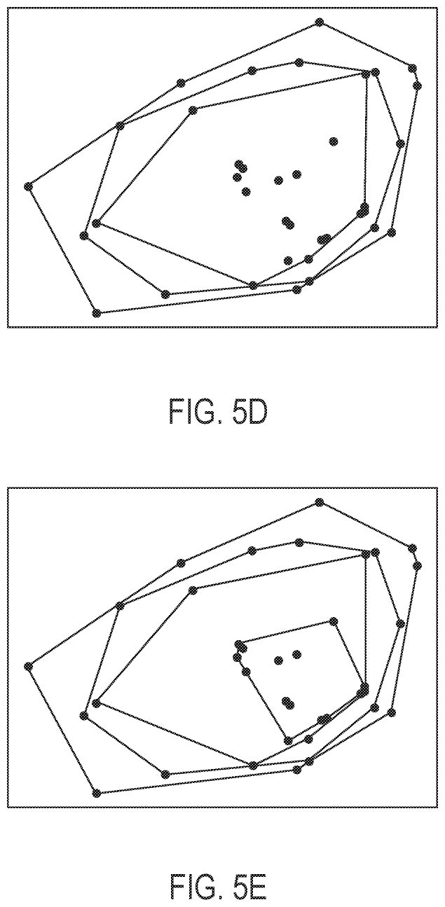

We solve this using an iterative heuristic that solves a simple assignment problem on each iteration. First, we define a convex hull as the smallest convex set of points containing all other points. Or, more simply, the smallest polygon (in terms of number of points) that contains all other points. FIGS. 4A and 4B show an example of a convex hull created from a random set of points. FIG. 4A shows an input set of points, under an embodiment. FIG. 4B shows a convex hull created from such set of points, under an embodiment.

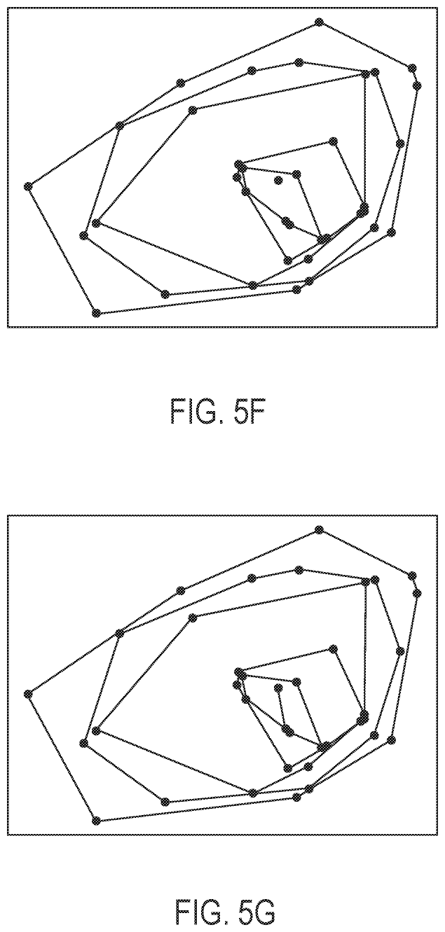

The heuristic works by dividing the set of points into hierarchical convex hulls, with each convex hull being the convex hull of all points contained within its parent convex hull. Then, working from the inside-out, an assignment problem is computed to connect inner convex hulls to outer convex hulls until there is a single cycle. FIGS. 5A-5F shows an example of generating convex hulls.

FIG. 5A shows a set of points on a 2-dimensional plane.

FIG. 5B shows an outer hull generated using all points shown in FIG. 5A.

FIG. 5C shows a second hull generated from the points left over in FIG. 5B.

FIG. 5D shows a third hull generated from the points left over in FIG. 5C.

FIG. 5E shows a fourth hull generated from the points left over in FIG. 5D.

FIG. 5F shows a fifth hull generated from the points left over in FIG. 5E.

FIG. 5G shows that the remaining points of FIG. 5F form the last hull.

### Iterative Hull Assignment

After the hierarchical hulls have been generated, an assignment problem is run to connect hulls, starting from the interior hull and working out. Assume we have a list of hulls, H, ordered such that the first item in H is the most interior of the hulls. We take the following steps: While length (H)>1:
Pop the first item from H and call this hull h₀. Let h₁ be the first item in H after h₀ is popped from the front of the list.
Run the assignment problem (described below) to connect h₀ and h₁. Call the new cycle created hₙ.
Replace h₁ in H with hₙ, so that hₙ is now the first item in H and h₁ no longer exists.
   There is now only one element in H. This is the final output from the heuristic.

### Assignment Problem

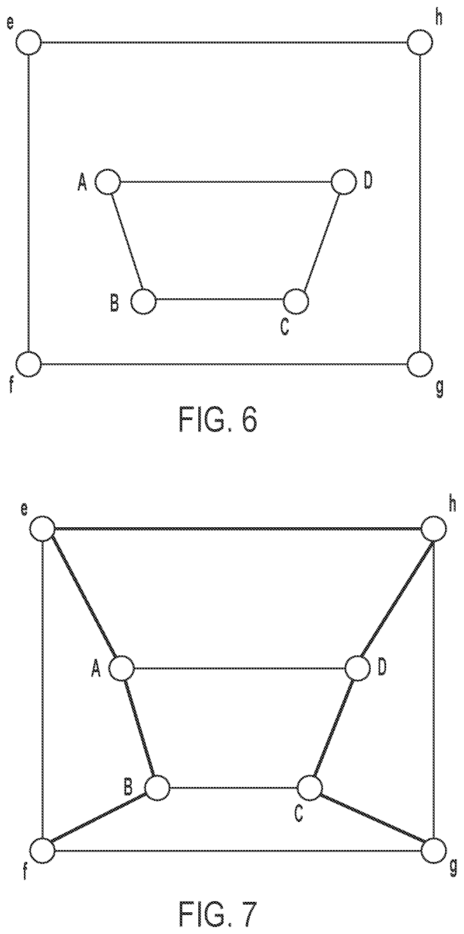

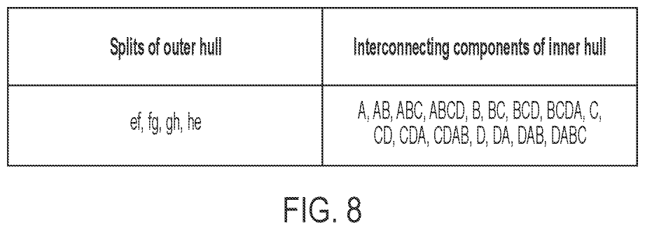

The assignment problem merges two hulls by “splitting” open legs of the outer hull and inserting portions of the inner hull into the splits. Consider the following example, with inner hull ABCD and outer hull efgh. FIG. 6 shows inner (ABCD) and outer (efgh) hulls created from 8 points. FIG. 7 shows that inner and outer hulls are connected by joining AB to the “break” between e and f, and by joining CD to the “break” created between g and h.

In FIG. 6, the hulls are shown unconnected. In FIG. 7, the shortest path is shown in red (or in bold). Note that segment AB of the inner hull was inserted into a split created between line segment ef. Likewise, segment CD was inserted into the split created between gh.

The assignment problem works by evaluating all potential splits and split insertions and choosing the minimum cost assignment such that each line segment in the inner hull is inserted into exactly one split in the outer hull. It is done in such a way that the order of points in the outer hull (efgh) remains the same as the order in the combined path, and the order of the points in the inner hull (ABCD) remains the same as their order in the combined path. This enables the problem to be solved in polynomial time.

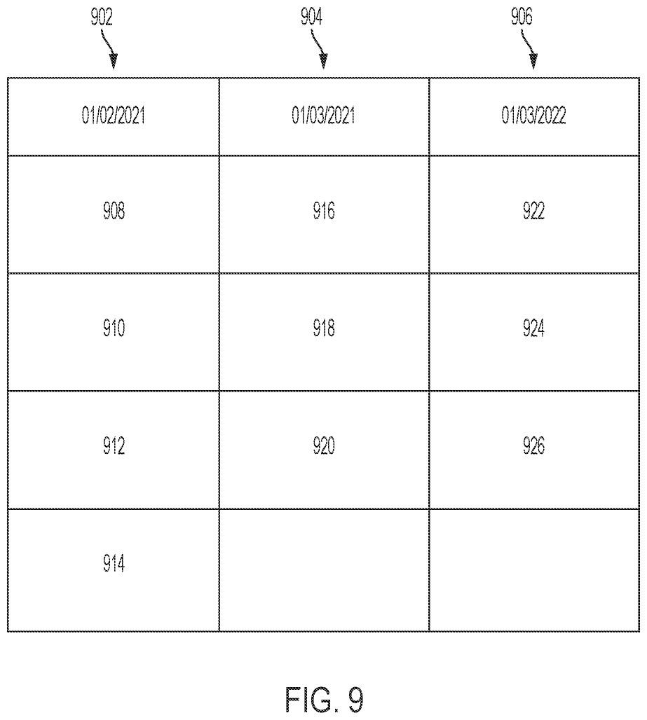

In FIG. 6, there are four possible splits of the outer hull and 16 possible interconnecting components of the inner hull as enumerated in FIG. 8 which shows possible splits and pairings of the inner and outer hull.

It should also be noted that each split of the outer hull is not required to be assigned any interconnecting parts of the inner hull.

The cost of each assignment is calculated as the length of the new path created minus the link removed from the outer hull. This is best demonstrated through an example. Consider the cost of adding leg BC to the split gh of the outer hull. The cost of this is the length of the new path created, [g, B, C, h], which is simply g̅B̅+B̅C̅+C̅h̅. In the case where no leg is added between split gh, the cost is simply g̅h̅.

The assignment problem then is:

### Parameters and Constants

c_{i,θ} Cost of assigning inner component i to outer splits θ.
I: Set of all possible inner components.
Θ: Set of all possible outer spits.
J: Set of all inner nodes.
Iⱼ: Set of all possible inner components that contain node.

### Decision Variables

x_{i,θ} Binary decision variable, 1 if inner component i is assigned to outer segment θ

### Minimize the Distance of the Assigned Connections

$$Minimize {\sum }_{θ ∈ Θ} {\sum }_{i ∈ I} {x}_{i , θ} * {c}_{i , θ} \qquad ( 2 g )$$

### Subject to:

All outer hull segments are connected to exactly one inner hull segment (including the null segment, which has a non-zero cost, which is the distance of the outer hull segment).

$${\sum }_{i ∈ I ⋃ ∅} {x}_{i , θ} = 1 ∀ θ ∈ Θ \qquad ( 2 h )$$

All inner hull nodes are connected to exactly one outer hull segment.

Σ_{θ∈Θ}Σ_{i∈I}_{ⱼ}x_{i,θ}=1∀j∈J (2i)

The binary decision variable determines whether inner segment i is assigned to outer break θ.

x_{i,θ}=0 or 1∀i∈I,θ∈Θ (2j)

### 5. Output Layer

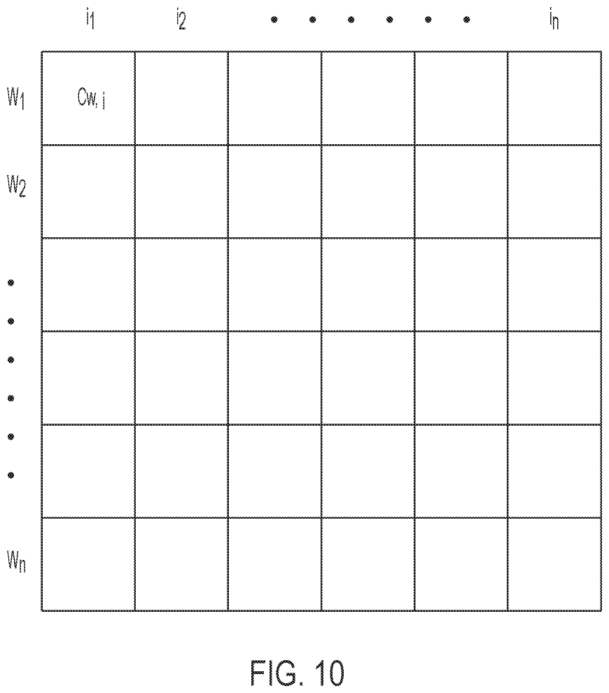

After completion of the algorithm, the output assignments and routes are output back into the home healthcare management database for presentation and acceptance by each office. The output reports viewed by the manual schedulers at each office are presented in calendar format, and may be color coded to indicate key details to the manual schedulers in case adjustments to the scheduler are required. FIG. 9 shows an example of electronic interface generated by the smart scheduler. FIG. 9 presents a clinician's scheduling information in calendar format. As seen in FIG. 9, a clinician is assigned to visits 910, 912, 914 on Jan. 2, 2020, to visits 918, 920 on Jan. 3, 2020, and to visits 924, 926 on Jan. 4, 2020. The interface may provide essential information for each visit. As on example, visit 910 may indicate time of day, type of visit, need for additional equipment, etc. FIG. 9 also presents a clinician with routing information for each day. The calendar entries 908, 916, 922 may present a clickable link to a map providing visual routing information for the corresponding day.

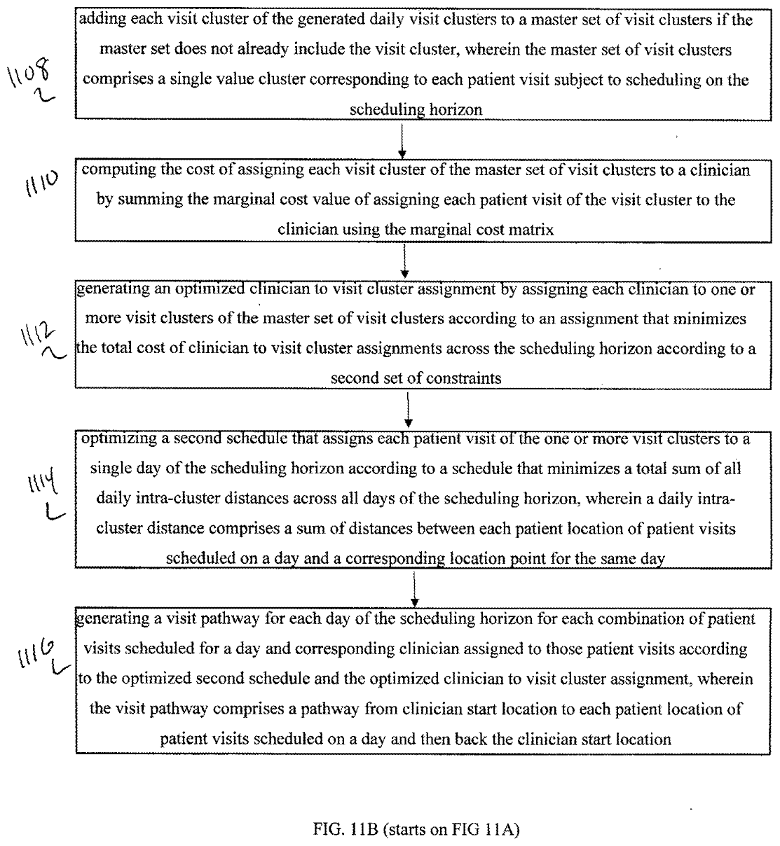

FIGS. 11A and 11B show a method for scheduling delivery of health care services, under an embodiment. The method includes 1102 generating a cost matrix, wherein each row of the cost matrix comprises a clinician, wherein each column of the cost matrix comprises a patient visit, wherein each patient visit comprises a patient, a patient visit type, a patient location, a productivity point value corresponding to an amount of work required of a clinician to complete the patient visit, and a scheduling window, wherein the scheduling window comprises days in a scheduling horizon when a patient is available to receive the corresponding patient visit, wherein the scheduling horizon comprises a plurality of future days subject to scheduling, wherein each entry in the cost matrix represents a clinician and patient visit combination, wherein each entry in the cost matrix comprises a marginal cost value of assigning a clinician to a patient visit, wherein the rows of the cost matrix represent all patient visits subject to scheduling in the scheduling horizon, wherein the columns represent clinicians subject to scheduling in the scheduling horizon. The method includes 1104 optimizing a first proposed schedule of all patient visits across days in the scheduling horizon subject to a first set of constraints, wherein each day corresponds to a daily productivity point capacity, wherein the daily productivity point capacity for a day comprises a total amount of work deliverable by at least one clinician of the clinicians on that day, wherein a daily productivity ratio comprises a sum of productivity point values corresponding to patient visits scheduled on a day divided by a corresponding daily productivity capacity, wherein the optimizing the first proposed schedule comprises selecting a schedule of all patient visits that minimizes the largest daily productivity ratio across the scheduling horizon. The method includes 1106 generating daily visit clusters for each day of the first proposed schedule, wherein the generating a daily visit cluster for a day comprises identifying for each individual patient visit scheduled on that day all combinations of patient visits also scheduled on that day sharing a common patient visit type with the individual patient visit, at a maximum distance value from the individual patient visit, and not greater than a size value. The method includes 1108 adding each visit cluster of the generated daily visit clusters to a master set of visit clusters if the master set does not already include the visit cluster, wherein the master set of visit clusters comprises a single value cluster corresponding to each patient visit subject to scheduling on the scheduling horizon. The method includes 1110 computing the cost of assigning each visit cluster of the master set of visit clusters to a clinician by summing the marginal cost value of assigning each patient visit of the visit cluster to the clinician using the marginal cost matrix. The method includes 1112 generating an optimized clinician to visit cluster assignment by assigning each clinician to one or more visit clusters of the master set of visit clusters according to an assignment that minimizes the total cost of clinician to visit cluster assignments across the scheduling horizon according to a second set of constraints. The method includes 1114 optimizing a second schedule that assigns each patient visit of the one or more visit clusters to a single day of the scheduling horizon according to a schedule that minimizes a total sum of all daily intra-cluster distances across all days of the scheduling horizon, wherein a daily intra-cluster distance comprises a sum of distances between each patient location of patient visits scheduled on a day and a corresponding location point for the same day. The method includes 1116 generating a visit pathway for each day of the scheduling horizon for each combination of patient visits scheduled for a day and corresponding clinician assigned to those patient visits according to the optimized second schedule and the optimized clinician to visit cluster assignment, wherein the visit pathway comprises a pathway from clinician start location to each patient location of patient visits scheduled on a day and then back the clinician start location.

A method is described herein that includes generating a cost matrix, wherein each row of the cost matrix comprises a clinician, wherein each column of the cost matrix comprises a patient visit, wherein each patient visit comprises a patient, a patient visit type, a patient location, a productivity point value corresponding to an amount of work required of a clinician to complete the patient visit, and a scheduling window, wherein the scheduling window comprises days in a scheduling horizon when a patient is available to receive the corresponding patient visit, wherein the scheduling horizon comprises a plurality of future days subject to scheduling, wherein each entry in the cost matrix represents a clinician and patient visit combination, wherein each entry in the cost matrix comprises a marginal cost value of assigning a clinician to a patient visit, wherein the rows of the cost matrix represent all patient visits subject to scheduling in the scheduling horizon, wherein the columns represent clinicians subject to scheduling in the scheduling horizon. The method includes optimizing a first proposed schedule of all patient visits across days in the scheduling horizon subject to a first set of constraints, wherein each day corresponds to a daily productivity point capacity, wherein the daily productivity point capacity for a day comprises a total amount of work deliverable by at least one clinician of the clinicians on that day, wherein a daily productivity ratio comprises a sum of productivity point values corresponding to patient visits scheduled on a day divided by a corresponding daily productivity capacity, wherein the optimizing the first proposed schedule comprises selecting a schedule of all patient visits that minimizes the largest daily productivity ratio across the scheduling horizon. The method includes generating daily visit clusters for each day of the first proposed schedule, wherein the generating a daily visit cluster for a day comprises identifying for each individual patient visit scheduled on that day all combinations of patient visits also scheduled on that day sharing a common patient visit type with the individual patient visit, at a maximum distance value from the individual patient visit, and not greater than a size value. The method includes adding each visit cluster of the generated daily visit clusters to a master set of visit clusters if the master set does not already include the visit cluster, wherein the master set of visit clusters comprises a single value cluster corresponding to each patient visit subject to scheduling on the scheduling horizon. The method includes computing the cost of assigning each visit cluster of the master set of visit clusters to a clinician by summing the marginal cost value of assigning each patient visit of the visit cluster to the clinician using the marginal cost matrix. The method includes generating an optimized clinician to visit cluster assignment by assigning each clinician to one or more visit clusters of the master set of visit clusters according to an assignment that minimizes the total cost of clinician to visit cluster assignments across the scheduling horizon according to a second set of constraints. The method includes optimizing a second schedule that assigns each patient visit of the one or more visit clusters to a single day of the scheduling horizon according to a schedule that minimizes a total sum of all daily intra-cluster distances across all days of the scheduling horizon, wherein a daily intra-cluster distance comprises a sum of distances between each patient location of patient visits scheduled on a day and a corresponding location point for the same day. The method includes generating a visit pathway for each day of the scheduling horizon for each combination of patient visits scheduled for a day and corresponding clinician assigned to those patient visits according to the optimized second schedule and the optimized clinician to visit cluster assignment, wherein the visit pathway comprises a pathway from clinician start location to each patient location of patient visits scheduled on a day and then back the clinician start location.

The first set of constraints includes a constraint that each patient visit is assigned to exactly one day in the scheduling horizon, under an embodiment.

The first set of constraints includes a constraint that patient visits are only assignable on days within a patient's corresponding scheduling window, under an embodiment.

The first set of constraints includes a constraint that two patient visits involving the same patient are not assignable to a same day, under an embodiment.

The second set of constraints includes each clinician being assigned a daily clinician productivity value equal to or less than the clinician's maximum daily productivity point capacity, under an embodiment.

The second set of constraints includes a condition that each visit of the one or more visit clusters is assigned to a single clinician, under an embodiment.

The second set of constraints includes a condition that if a patient visit is assigned to a clinician, then a visit cluster of the one or more visit clusters including the same patient visit is also assigned to the same clinician, under an embodiment.

The second set of constraints includes a condition that a patient visit is only assignable to a clinician with the skill set necessary to complete the patient visit, under an embodiment.

The optimizing the second schedule of an embodiment comprises creating a minimal bounding box around geographic coordinates of patient locations for all patient visits and clinician locations for all clinicians subject to scheduling across the scheduling horizon, under an embodiment.

The optimizing the second schedule of an embodiment includes randomly assigning a location point within the bounding box for each day in the scheduling horizon, under embodiment.

The optimizing includes scheduling each patient visit of the one or more visit clusters according to a schedule that minimizes a total sum of all daily intra-cluster distances across all days of the scheduling horizon relative to the randomly assigned location points, under an embodiment.

The optimizing the second schedule includes updating each location point corresponding to each day of the scheduling horizon to be the geometric median of patient locations for patient visits scheduled on that day, under an embodiment.

The optimizing includes iteratively scheduling each patient visit of the one or more visit clusters according to a schedule that minimizes a total sum of all daily intra-cluster distances across all days of the scheduling horizon relative to the most recently updated location points, under an embodiment.

The iteratively scheduling ceases when the updated patient locations remain unchanged when compared to the immediately prior updated location points, under an embodiment.

A marginal cost value for an entry in the cost matrix comprises labor cost of assigning clinician to patient, under an embodiment.

A marginal cost value for an entry in the cost matrix comprises travel cost of assigning clinician to patient, under an embodiment.

A marginal cost value includes a continuity of care penalty for each patient and clinician combination of the cost matrix, under an embodiment.

The generating the visit pathway comprises generating a convex hull around patient locations for patient visits and corresponding clinician location, under an embodiment.

The generating the visit pathway comprises iteratively generating convex hulls around locations remaining within the previously generated convex hull, under an embodiment.

The generating the visit pathway comprises iteratively combining a most interior hull with a next most interior hull to create a new single hull, under an embodiment.

The creating the new single hull comprises minimizing distance of segments from an interior hull added to an outer hull, under an embodiment.

The iteratively combining ceases when a single remaining pathway is created, under an embodiment.

Computer networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network may be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network may include one of more LANs, WANs, Internets, and computers. The computers may serve as servers, clients, or a combination thereof.

The systems and methods for scheduling delivery of healthcare services can be a component of a single system, multiple systems, and/or geographically separate systems. The systems and methods for scheduling delivery of healthcare services can also be a subcomponent or subsystem of a single system, multiple systems, and/or geographically separate systems. The components of systems and methods for scheduling delivery of healthcare services can be coupled to one or more other components (not shown) of a host system or a system coupled to the host system.

One or more components of the systems and methods for scheduling delivery of healthcare services and/or a corresponding interface, system or application to which the systems and methods for scheduling delivery of healthcare services is coupled or connected includes and/or runs under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that include the systems and methods for scheduling delivery of healthcare services can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the systems and methods for scheduling delivery of healthcare services and corresponding systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the systems and methods for scheduling delivery of healthcare services and corresponding systems and methods include: microcontrollers with memory (such as electronically erasable programmable read only memory (EEPROM)), embedded microprocessors, firmware, software, etc. Furthermore, aspects of the systems and methods for scheduling delivery of healthcare services and corresponding systems and methods may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components may be processed by a processing entity (e.g., one or more processors) within the computer system in conjunction with execution of one or more other computer programs.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the systems and methods for scheduling delivery of healthcare services is not intended to be exhaustive or to limit the systems and methods to the precise forms disclosed. While specific embodiments of, and examples for, the systems and methods for scheduling delivery of healthcare services and corresponding systems and methods are described herein for illustrative purposes, various equivalent modifications are possible within the scope of the systems and methods, as those skilled in the relevant art will recognize. The teachings of the systems and methods for scheduling delivery of healthcare services and corresponding systems and methods provided herein can be applied to other systems and methods, not only for the systems and methods described above.

The elements and acts of the various embodiments described above can be combined to provide further embodiments. These and other changes can be made to the systems and methods for scheduling delivery of healthcare services and corresponding systems and methods in light of the above detailed description.

We claim:

1. A method, comprising
generating a cost matrix, wherein each row of the cost matrix comprises a clinician, wherein each column of the cost matrix comprises a patient visit, wherein each patient visit comprises a patient, a patient visit type, a patient location, a productivity point value corresponding to an amount of work required of a clinician to complete the patient visit, and a scheduling window, wherein the scheduling window comprises days in a scheduling horizon when a patient is available to receive the corresponding patient visit, wherein the scheduling horizon comprises a plurality of future days subject to scheduling, wherein each entry in the cost matrix represents a clinician and patient visit combination, wherein each entry in the cost matrix comprises a marginal cost value of assigning a clinician to a patient visit, wherein the rows of the cost matrix represent all patient visits subject to scheduling in the scheduling horizon, wherein the columns represent clinicians subject to scheduling in the scheduling horizon;
optimizing a first proposed schedule of all patient visits across days in the scheduling horizon subject to a first set of constraints, wherein each day corresponds to a daily productivity point capacity, wherein the daily productivity point capacity for a day comprises a total amount of work deliverable by at least one clinician of the clinicians on that day, wherein a daily productivity ratio comprises a sum of productivity point values corresponding to patient visits scheduled on a day divided by a corresponding daily productivity capacity, wherein the optimizing the first proposed schedule comprises selecting a schedule of all patient visits that minimizes the largest daily productivity ratio across the scheduling horizon;
generating daily visit clusters for each day of the first proposed schedule, wherein the generating a daily visit cluster for a day comprises identifying for each individual patient visit scheduled on that day all combinations of patient visits also scheduled on that day sharing a common patient visit type with the individual patient visit, at a maximum distance value from the individual patient visit, and not greater than a size value;
adding each visit cluster of the generated daily visit clusters to a master set of visit clusters if the master set does not already include the visit cluster, wherein the master set of visit clusters comprises a single value cluster corresponding to each patient visit subject to scheduling on the scheduling horizon;
computing the cost of assigning each visit cluster of the master set of visit clusters to a clinician by summing the marginal cost value of assigning each patient visit of the visit cluster to the clinician using the marginal cost matrix;
generating an optimized clinician to visit cluster assignment by assigning each clinician to one or more visit clusters of the master set of visit clusters according to an assignment that minimizes the total cost of clinician to visit cluster assignments across the scheduling horizon according to a second set of constraints;
optimizing a second schedule that assigns each patient visit of the one or more visit clusters to a single day of the scheduling horizon according to a schedule that minimizes a total sum of all daily intra-cluster distances across all days of the scheduling horizon, wherein a daily intra-cluster distance comprises a sum of distances between each patient location of patient visits scheduled on a day and a corresponding location point for the same day; and
generating a visit pathway for each day of the scheduling horizon for each combination of patient visits scheduled for a day and corresponding clinician assigned to those patient visits according to the optimized second schedule and the optimized clinician to visit cluster assignment, wherein the visit pathway comprises a pathway from clinician start location to each patient location of patient visits scheduled on a day and then back the clinician start location.

2. The method of claim 1, wherein the first set of constraints includes a constraint that each patient visit is assigned to exactly one day in the scheduling horizon.

3. The method of claim 1, wherein the first set of constraints includes a constraint that patient visits are only assignable on days within a patient's corresponding scheduling window.

4. The method of claim 1, wherein the first set of constraints includes a constraint that two patient visits involving the same patient are not assignable to a same day.

5. The method of claim 1, wherein the second set of constraints includes each clinician being assigned a daily clinician productivity value equal to or less than the clinician's maximum daily productivity point capacity.

6. The method of claim 1, wherein the second set of constraints includes a condition that each visit of the one or more visit clusters is assigned to a single clinician.

7. The method of claim 1, wherein the second set of constraints includes a condition that if a patient visit is assigned to a clinician, then a visit cluster of the one or more visit clusters including the same patient visit is also assigned to the same clinician.

8. The method of claim 1, wherein the second set of constraints includes a condition that a patient visit is only assignable to a clinician with the skill set necessary to complete the patient visit.

9. The method of claim 1, the optimizing the second schedule comprising creating a minimal bounding box around geographic coordinates of patient locations for all patient visits and clinician locations for all clinicians subject to scheduling across the scheduling horizon.

10. The method of claim 9, the optimizing the second schedule including randomly assigning a location point within the bounding box for each day in the scheduling horizon.

11. The method of claim 10, the optimizing including scheduling each patient visit of the one or more visit clusters according to a schedule that minimizes a total sum of all daily intra-cluster distances across all days of the scheduling horizon relative to the randomly assigned location points.

12. The method of claim 11, the optimizing the second schedule including updating each location point corresponding to each day of the scheduling horizon to be the geometric median of patient locations for patient visits scheduled on that day.

13. The method of claim 12, the optimizing including iteratively scheduling each patient visit of the one or more visit clusters according to a schedule that minimizes a total sum of all daily intra-cluster distances across all days of the scheduling horizon relative to the most recently updated location points.

14. The method of claim 13, wherein the iteratively scheduling ceases when the updated patient locations remain unchanged when compared to the immediately prior updated location points.

15. The method of claim 1, wherein a marginal cost value for an entry in the cost matrix comprises labor cost of assigning clinician to patient.

16. The method of claim 1, wherein a marginal cost value for an entry in the cost matrix comprises travel cost of assigning clinician to patient.

17. The method of claim 1, wherein a marginal cost value includes a continuity of care penalty for each patient and clinician combination of the cost matrix.

18. The method of claim 1, the generating the visit pathway comprising generating a convex hull around patient locations for patient visits and corresponding clinician location.

19. The method of claim 18, the generating the visit pathway comprising iteratively generating convex hulls around locations remaining within the previously generated convex hull.

20. The method of claim 19, the generating the visit pathway comprising iteratively combining a most interior hull with a next most interior hull to create a new single hull.

21. The method of claim 20, wherein the creating the new single hull comprises minimizing distance of segments from an interior hull added to an outer hull.

22. The method of claim 21, wherein the iteratively combining ceases when a single remaining pathway is created.
